(19) United States
(12) Patent Application Publication
(10) Pub. No.: US 2026/0283852 A1
(43) Pub. Date: Sep. 24, 2026

(54) HEAD-WORN OPTICAL PROTECTION DEVICE WITH LENS CLAMP ASSEMBLY

(71) Applicant: IPG PHOTONICS CORPORATION, Marlborough, MA (US)

(72) Inventor: Brett Ruggles, Marlborough, MA (US)

(21) Appl. No.: 19/677,641

(22) Filed: May 14, 2026

Related U.S. Application Data
(63) Continuation of application No. PCT/US2024/056126, filed on Nov. 15, 2024.
(60) Provisional application No. 63/599,597, filed on Nov. 16, 2023.

(51) Int. Cl. A61F 9/06

(52) U.S. Cl. CPC A61F 9/065

(57) ABSTRACT

A head-worn optical protection device includes a lens clamp assembly that facilitates adding and/or removing at least one of a plurality of lenses. The head-worn optical protection device may be a welding helmet particularly suited for laser welding applications. The lens clamp assembly includes one or more lens clamps that engage one or more lenses and holds the lens(es) against a lens frame in a main body of the protection device. In one embodiment, the lens clamp assembly may include a releasable lens clamp with an actuator that releasably engages the lens frame and facilitates removal of lenses in a lens stack. In another embodiment, the lens clamp assembly may include an outer lens clamp for securing an impact shielding lens in an outer portion of the lens frame and a separate inner lens clamp for securing optical lenses, such as DIN-rated lenses, in an inner portion of the lens frame.

### CROSS-REFERENCE TO RELATED APPLICATIONS

The present application is a continuation of PCT Application No. PCT/US2024/056126 filed Nov. 15, 2024, which claims the benefit of U.S. Provisional Application Ser. No. 63/599,597, filed Nov. 16, 2023, both of which are fully incorporated herein by reference.

### TECHNICAL FIELD

The present disclosure relates to head-worn optical protection devices and more particularly, to a head-worn optical protection device with a lens clamp assembly to facilitate replacing lenses.

### BACKGROUND INFORMATION

Head-worn optical protection devices are often worn to protect the eyes and other parts of the face and head from injury during certain potentially hazardous activities. One example of such an optical protection device is a welding helmet worn by welders to provide optical protection from welding activities. Welding helmets include protective lenses that protect the eyes from intense full-spectrum light rays as well as sparks emitted by a welding tool and debris generated by the welding process. Existing welding helmets have certain disadvantages, however, particularly for laser welding applications, in regards to their protection against infrared light.

One disadvantage with existing welding helmets is weight. Enhancing the optical protection to account for infrared light may necessitate adding lens components and fixtures, to a point of excessive weight. This prevents the existing welding helmets from being worn comfortably and may interfere with the overall safety of the welding process. Simply reducing the envelope of the helmet to reduce weight without sacrificing functionality presents some challenges. Laser welding applications, in particular, call for a more complex stack of lenses to be mounted in the helmet. This is to account for front impact resistance, direct IR protection, off-angle IR light protection, as well as different DIN-rated lenses that may be needed for visible light. Existing welding helmets do not allow for lenses to be easily swapped out for such applications.

### SUMMARY

According to one aspect of the present disclosure, a head-worn optical protection device includes a main body defining a window, headgear for securing the main body to a head of a user, and a lens frame around the window in the main body. The lens frame defines first and second frame sides and first and second frame pockets located in the first and second frame sides, respectively. The head-worn optical protection device also includes a lens stack including at least one lens and a lens clamp assembly for releasably clamping the lens stack to the lens frame in the window of the main body. The lens clamp assembly includes a lens clamp having first and second clamp sides engaging the first and second frame pockets for clamping the lens stack in the lens frame.

According to another aspect of the present disclosure, a head-worn optical protection device includes a main body defining a window, headgear for securing the main body to a head of a user, and a lens frame around the window in the main body. The lens frame has an outer portion extending through the window of the main body and an inner portion on an inside of the main body. The head-worn optical protection device also includes at least an impact shielding lens secured in the outer portion of the lens frame, a lens stack secured in the inner portion of the lens frame, and a lens clamp assembly configured to secure the impact shielding lens and the lens stack in the lens frame.

### BRIEF DESCRIPTION OF THE DRAWINGS

These and other features and advantages will be better understood by reading the following detailed description, taken together with the drawings wherein:

FIG. 1A is an exploded view of a head-worn optical protection device including a lens clamp assembly, consistent with embodiments of the present disclosure.

FIG. 1B is a perspective view of the assembled head-worn optical protection device shown in FIG. 1A.

FIGS. 2A and 2B are perspective views of a lens frame used in the head-worn optical protection device of FIG. 1A.

FIG. 2C is a plan view of the lens frame shown in FIGS. 2A and 2B.

FIG. 2D is a cross-sectional view of the lens frame taken along line A-A in FIG. 2C.

FIG. 2E is a cross-sectional view of the lens frame taken along line B-B in FIG. 2C.

FIG. 3A is a lens clamp used in the head-worn optical protection device of FIG. 1A.

FIG. 3B is a top plan view of the lens clamp shown in FIG. 3A.

FIG. 3C is a cross-sectional view of the lens clamp taken along line A-A in FIG. 3B.

FIG. 3D is a bottom plan view of the lens clamp shown in FIG. 3A.

FIG. 4A is a perspective view of a gasket used in the head-worn optical protection device of FIG. 1.

FIG. 4B is a plan view of the gasket shown in FIG. 4A.

FIGS. 5A and 5B are perspective views of an actuating clip used in the head-worn optical protection device of FIG. 1.

FIG. 5C is a side view of the actuating clip shown in FIGS. 5A and 5B.

FIG. 5D is an outside view of the actuating clip shown in FIGS. 5A and 5B.

FIG. 5E is an inside view of the actuating clip shown in FIGS. 5A and 5B.

FIGS. 6A and 6B are perspective views inside the head-worn optical protection device of FIG. 1B showing the lens clamp assembly being moved to a clamped position.

FIG. 6C is a cross-sectional view of the lens clamp assembly in the clamped position inside of the optical protection device.

FIG. 6D is a close-up view of the lens clamp assembly in the seated or clamped position.

FIG. 6E is a close-up view of the lens clamp assembly in the released position and actuated for removal.

FIG. 7A is an exploded view of a head-worn optical protection device including a lens clamp assembly, consistent with another embodiment of the present disclosure.

FIG. 7B is an enlarged view of an outer lens clamp for securing an impact shielding lens to a lens frame in the head-worn optical protection device shown in FIG. 7A.

### DETAILED DESCRIPTION

A head-worn optical protection device, consistent with embodiments of the present disclosure, includes a lens clamp assembly that facilitates adding and/or removing at least one of a plurality of lenses. The head-worn optical protection device may be a welding helmet particularly suited for laser welding applications. The lens clamp assembly includes one or more lens clamps that engage one or more lenses and hold the lenses against a lens frame in a main body of the protection device. In one embodiment, the lens clamp assembly may include a releasable lens clamp with a releasable actuator that releasably engages the lens frame and facilitates removal of lenses in a lens stack including an impact shielding lens and optical lenses such as DIN-rated lenses. In another embodiment, the lens clamp assembly may include an outer lens clamp for securing an impact shielding lens in an outer portion of the lens frame and a separate inner lens clamp for securing the optical lenses, such as DIN-rated lenses, in an inner portion of the lens frame.

As used herein, the term “lens” refers to a piece of transparent material that allows light to pass through and the term “filter” refers to a lens that modifies the light passing through. The term “lens stack” refers to one or more lenses and/or filters arranged together in a stack.

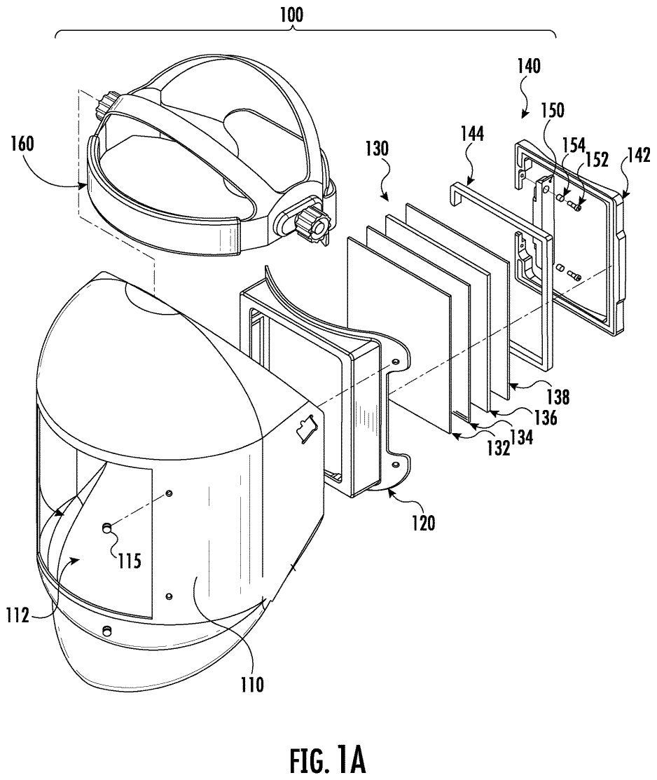

Referring to FIGS. 1A and 1B, a head-worn optical protection device 100, consistent with embodiments of the present disclosure, is described in greater detail. In the illustrated embodiment, the head-worn optical protection device 100 is a welding helmet designed for laser welding applications. A head-worn optical protection device using the concepts described herein may also be designed for other welding applications or for other optical protection applications.

The head-worn optical protection device 100 includes a main body 110, a lens frame 120 positioned in a window 112 defined by the main body 110, a lens stack 130 supported and framed by the lens frame 120, and a lens clamp assembly 140 releasably clamping the lens stack 130 to the lens frame 120 inside the main body 110. In this embodiment, the lens clamp assembly 140 includes a releasable lens clamp 142, a gasket 144 positioned against a face of the lens clamp 142, and an actuating clip 150 on one side of the lens clamp 142. The actuating clip 150 may be secured to the lens clamp 142 using one or more fasteners 152 and springs 154 to allow the actuating clip 150 to be moved for actuating the releasable lens clamp 142, as will be described in greater detail below. The head-worn optical protection device 100 also includes headgear 160 secured to the main body 110 for mounting on the head of a user.

The main body 110 may be in the form of a helmet shell and may extend around the sides of the head, the top of the head and down to the neck, although other configurations and shapes are contemplated. In the illustrated embodiment, the lens frame 120, the lens stack 130, and the lens clamp assembly 140 together form a lens box 114 that at least partially extends through and protrudes from the window 112 of the main body 110 (see FIG. 1B). In other embodiments, the lens box 114 may be substantially flush with the front of the main body 110. The main body 110 may be made of carbon fiber, which provides a good strength-to-weight ratio, although other materials may also be used. The headgear 160 may include adjustable headgear such as the type known for use in welding helmets. Although one example of the headgear 160 is shown, other types of headgear 160 may be used.

The lens stack 130 may include one or more lenses and/or filters providing various types of physical and optical protection. In an embodiment, the lens stack 130 may include, for example, an impact shielding lens 132, an IR protective lens 134, a filter 136, and a DIN-rated lens 138 (e.g., a DIN-rated weld shade). The impact shielding lens 132 may include, for example, a clear and completely transparent polycarbonate lens that provides impact resistance. The IR protective lens 134 may include, for example, an IR-reflective glass pane, which is able to reflect infrared (IR) light away from the wearer while also being durable enough to withstand the heat-load from direct exposure to laser light. The filter 136 may include, for example, an acrylic absorptive pane directly behind the reflective glass pane, which provides additional protection in the event of possible high angle of incidence (AOI) or “off-angle” light. Instead of the filter 136, an absorptive coating may be applied, for example, to the IR protective lens 134 and/or the DIN-rated lens 138.

The DIN rated lens 138 may be selected depending upon the particular application. In some embodiments, the DIN rated lens 138 may include a polycarbonate lens with a DIN rating of 3 to 13 and more specifically, a DIN rating of 3 to 6 for laser welding applications, such as a DIN 3 polycarbonate lens or a DIN 5 polycarbonate lens. The DIN rating may be selected based on the plasma intensity of the welded material. Other lenses may also be used, such as an auto-dimming adjustable DIN shade, although the polycarbonate DIN-rated lens is advantageously lighter. In the illustrated embodiment, the lens clamp assembly 140 allows any one or all of the lenses 132, 134, 136, 138 in the lens stack 130 to be easily replaced or swapped out for different applications or if damaged.

The lens stack 130 may include an integrated stack of lenses or a single lens designed to provide the desired mechanical and optical protection. Two or more of the lenses 132, 134, 136, 138 may be bonded together, for example, using a mechanical jacket or epoxy, to create one large, layered lens. In one embodiment, the IR protective lens 134 and the filter 136 may be bonded together with the impact shielding lens 132 with the DIN rated lens 138 being decoupled. Decoupling the impact shielding lens 132 may be advantageous because this component may be consumed more frequently and require replacement. Decoupling the DIN rated lens 138 may be advantageous to allow different DIN rated lenses to be swapped out for different applications.

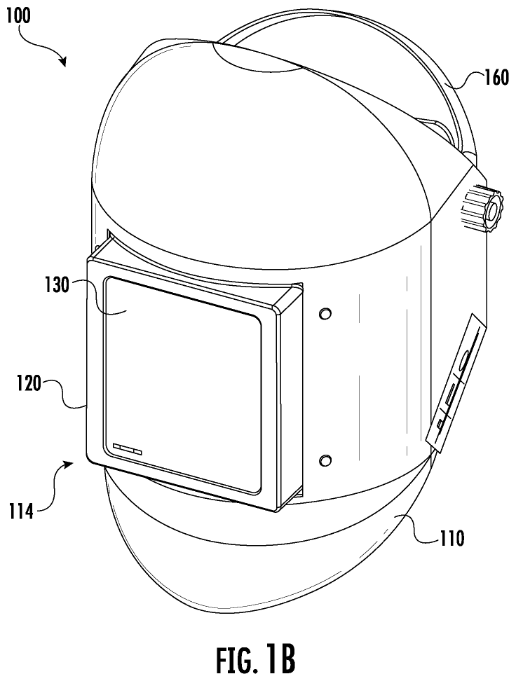

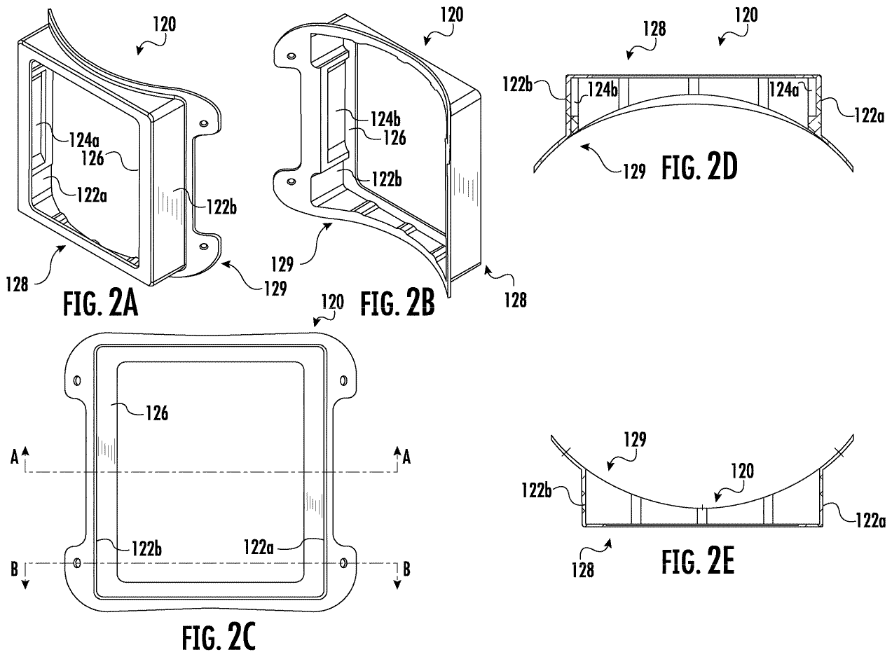

Referring to FIGS. 2A-2E, an embodiment of the lens frame 120 is described in greater detail. The lens frame 120 includes sides 122a, 122b defining pockets 124a, 124b that receive protruding edges (described below) of the lens clamp 142 and the actuating clip 156. The frame 120 also includes a mounting surface 126 that supports the lens stack 130 such that the lens stack 130 is held between the lens clamp 142 and the mounting surface 126. In the illustrated embodiment, an outer portion 128 of the frame 120 extends through the window 112 of the main body 110 providing a protruding lens box design and an inner portion 129 of the lens frame 120 is curved (FIGS. 2D and 2E) to conform generally with a face of the user wearing the device. The protruding lens box design may accommodate a lens stack 130 with multiple lenses while providing clearance inside of the optical protection device 100. In other embodiments, the outer portion 128 of the frame 120 may be substantially flush with the outer surface of the main body 110.

The lens frame 120 may be made of 6061 aluminum or other suitable material and may be mounted inside of the main body 110 by securing the inner portion 129 to the main body 110 using mounting fasteners, such as rivets 115 (shown in FIG. 1). Although the inner portion 129 of the lens frame 120 is mounted to the main body 110 with fasteners (e.g., rivets 115) in the illustrated embodiment, the lens frame 120 may also be mounted in other ways or formed integrally with or molded together with the main body 110, for example, from carbon fiber material. Other designs and configurations for the frame 120 are also contemplated.

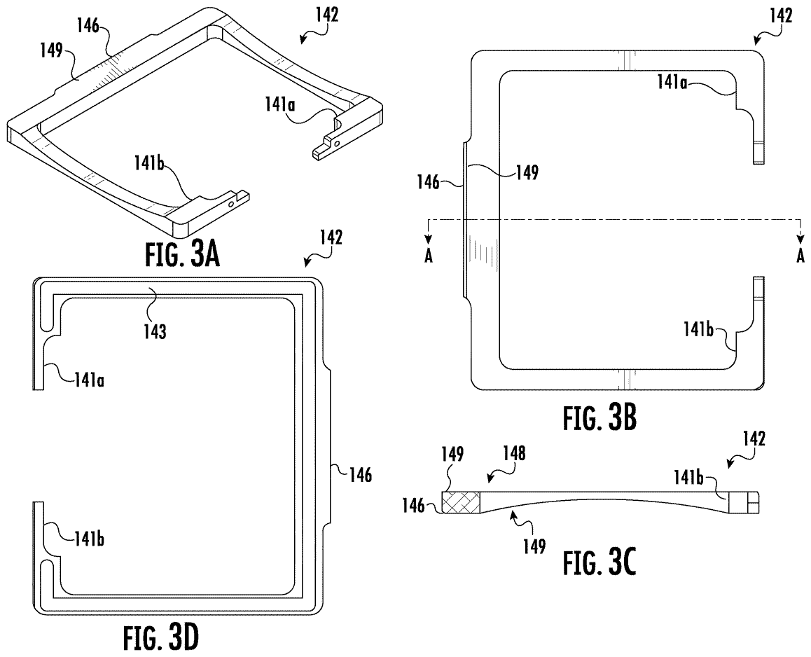

Referring to FIGS. 3A-3D, an embodiment of the releasable lens clamp 142 is described in greater detail. The lens clamp 142 is generally C-shaped and designed to engage and hold the lens stack 130 against the mounting surface 126. The lens clamp 142 includes arms 141a, 141b that define an open region for attaching the actuating clip 150 between the arms 141a, 141b. The lens clamp 142 includes a fixed protruding edge 146 extending from a side of the lens clamp 142, which provides a lead-in for engaging one of the pockets 124a, 124b in the lens frame 120. The fixed protruding edge 146 provides a ledge 149 that engages an underside ledge defined in one of the pockets 124a, 124b of the lens frame 120. A front face 148 of the lens clamp 142 is generally flat and includes a groove 143 for receiving the gasket 144. A rear face 149 of the lens clamp 142 may be curved to conform generally with the face of the user. The lens clamp 142 may be made of 6061 aluminum or other suitable material. Other designs and configurations for the lens clamp 142 are also contemplated.

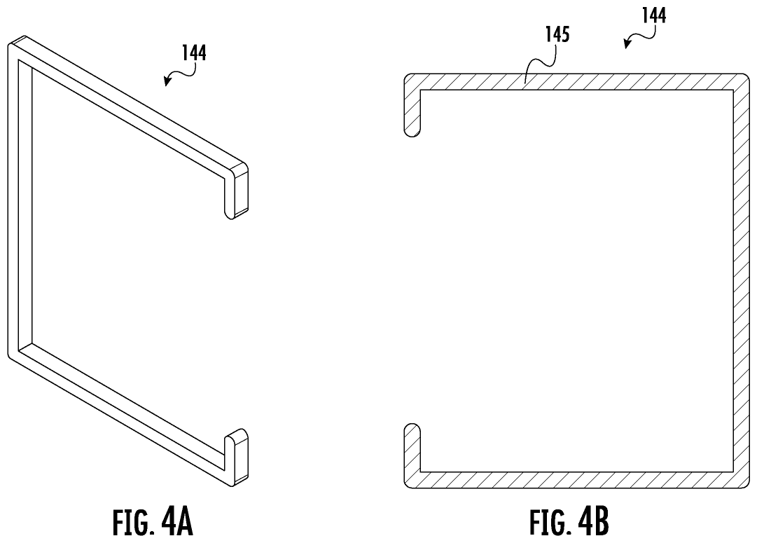

Referring to FIGS. 4A and 4B, an embodiment of the gasket 144 is described in greater detail. The gasket 144 may have a C-shape similar to the clamp 142 such that the gasket 144 fits in the groove 143 on the front face 148 of the clamp 142. An adhesive 145 may be used to secure the gasket 144 within the groove 143. The gasket 144 may be made of a resilient material such as a foam, and more specifically, a relatively soft foam (e.g., 3 psi @25% compression). The relatively soft foam material allows enough flexibility to accommodate and securely clamp different lens stacks with different thicknesses (e.g., different numbers of lenses and/or thicknesses of the lenses) and thus accounts for any unexpected differences in thickness due to the tolerance stack-up of each individual lens.

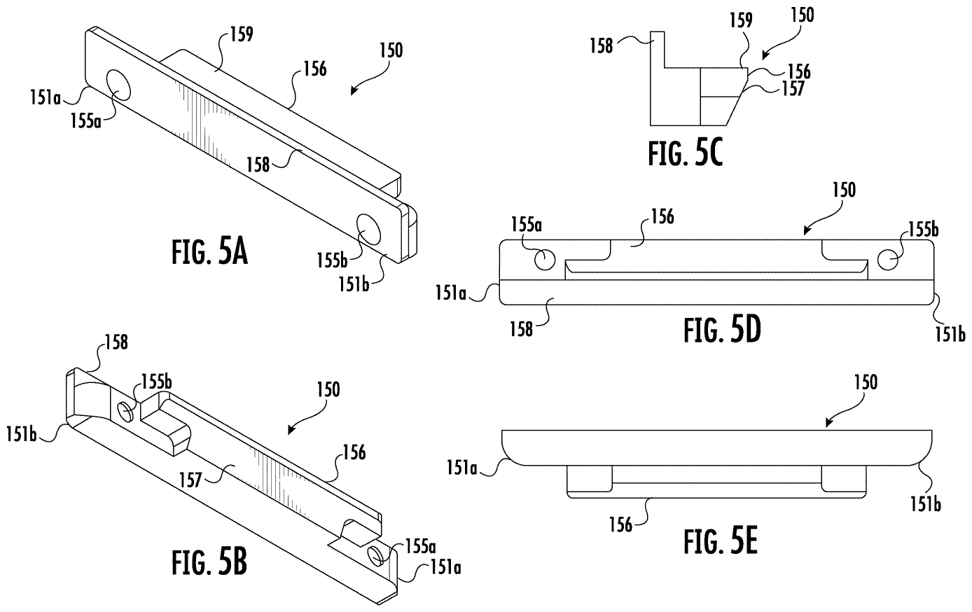

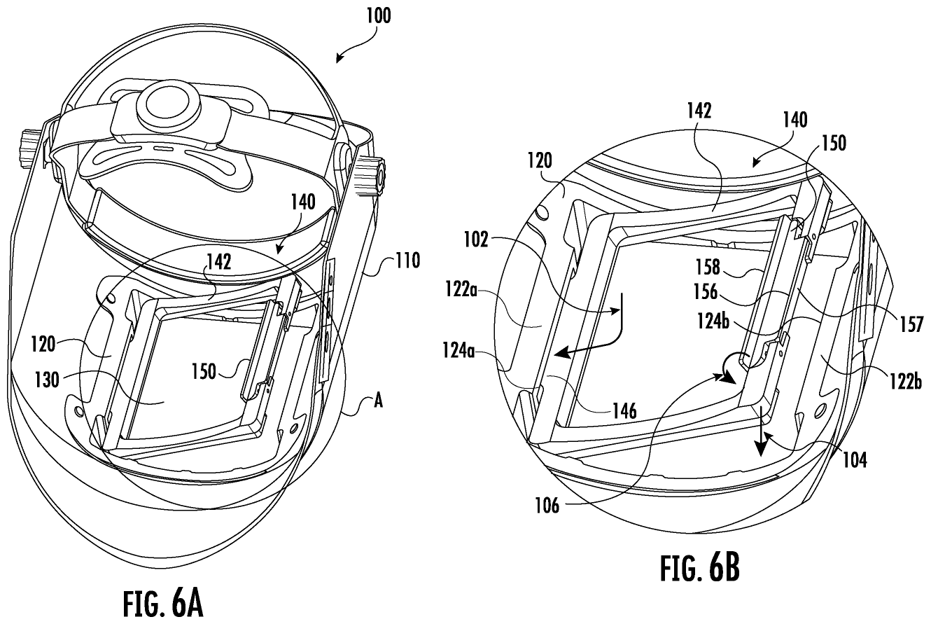

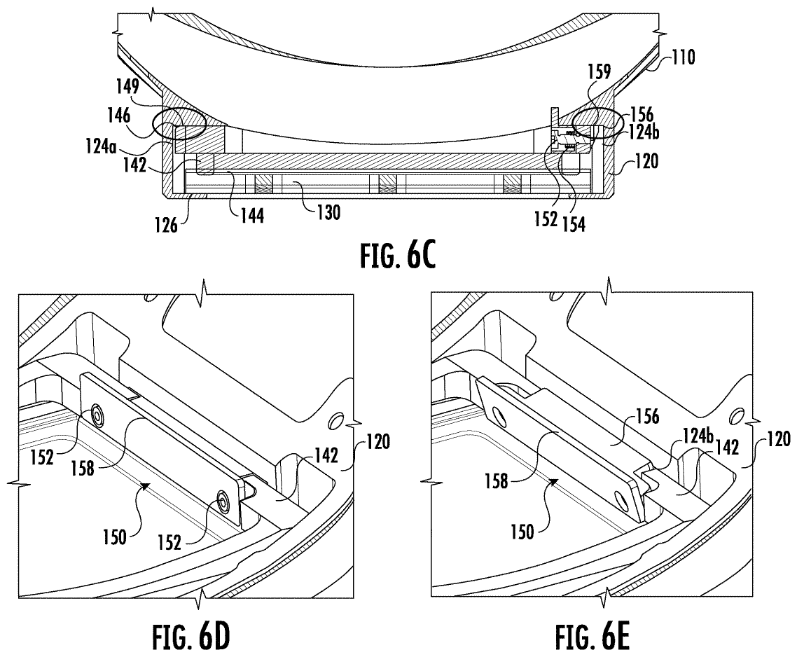

Referring to FIGS. 5A-5E, an embodiment of the actuating clip 150 is described in greater detail. The actuating clip 150 includes ends 151a, 151b shaped and configured to fit between and be secured to the arms 141a, 141b of the lens clamp 142. The actuating clip 150 includes apertures 155a, 155b extending through the ends 151a, 151b for receiving the fasteners 152 and springs 154, as shown in FIG. 1A. The fasteners 152 secure each of the ends 151a, 151b to the respective arms 141a, 141b of the lens clamp 142 with springs 154 between the fasteners 152 and the actuating clip 150 (see FIGS. 6C and 6D). The apertures 155a, 155b have a dimension that allows the actuating clip 150 to pivot relative to the lens clamp 142 from the clamped position to the released position while remaining secured to the lens clamp 142. The actuating clip 150 may be made of 6061 aluminum or other suitable material. Other designs and configurations for the actuating clip 150 are also contemplated.

The actuating clip 150 includes a movable protruding edge 156 that is located in the open region defined by the arms 141a, 141b when the actuating clip 150 is secured to the arms 141a, 141b. The movable protruding edge 156 moves with the actuating clip 150 to engage the other of the pockets 124a, 124b in the lens frame 120. The movable protruding edge 156 may include an angled surface 157 that slides over the lens frame 120 when the lens clamp 142 is moved to the clamped position (see FIG. 6B), thereby facilitating the clamping operation. The movable protruding edge 156 also provides a ledge 159 that engages an underside ledge defined in one of the pockets 124a, 140b in the lens frame 120. The movable protruding edge 156 and the fixed protruding edge 146 and the pockets 124a, 124b may have substantially the same or similar dimensions to provide symmetry of the lens clamp assembly 140 about the vertical axis of the device to allow the clamp assembly 140 to be rotated 180°about the fore-aft direction without sacrificing any functionality.

The actuating clip 150 further includes a tab 158 that may be grasped by the user to actuate the actuating clip 150. The fasteners 152 may include threaded fasteners, such as shoulder screws, or any other fasteners capable of securing the actuating clip 150 to the lens clamp 142. The springs 154 bias the actuating clip 150 into the clamped position and may include helical springs or any other spring elements capable of biasing the actuating clip 150. The fasteners 152 and springs 154 facilitate consistent actuation and accurate retention of the actuating clip 150.

FIGS. 6A-6E illustrate operation of the lens clamp assembly 140. As shown in FIG. 6A, the lens stack 130 is positioned inside the lens frame 120 and the lens clamp assembly 140 is engaged with the lens frame 120 to secure the lens stack 130. In particular, the fixed protruding edge 146 of the releasable lens clamp 142 is first inserted into the pocket 124a in the lead-in direction shown by arrow 102. The lens clamp 142 is then moved downward in the direction of arrow 104 while the actuating clip 150 is actuated by rotating the actuating clip 150 counter-clockwise in an upward direction as shown by arrow 106 to allow the movable protruding edge 156 of the actuating clip 150 to be inserted into the opposite pocket 124b of the lens frame 120. The angled surface 157 of the actuating clip 150 may engage and slide over the side 122b of the lens frame 120 as the lens clamp 142 and the actuating clip 150 move to the clamped position, thereby facilitating rotation of the actuating clip 150 and engagement with the pocket 124b. Thus, the actuating clip 150 may be rotated and actuated by the user grasping the tab 158 and/or by the engagement of the actuating clip 150 with the lens frame 120. When the lens clamp 142 is firmly secured against the rear-most lens (e.g., lens 138) of the lens stack 130, the moveable protruding edge 156 of the actuating clip 150 enters the pocket 124b and rotates back to the resting or engaged position, fully-seated in the pocket 124b.

Although an orientation of the lens clamp assembly 140 is shown with the actuating clip 150 engaging the pocket 124b on the right side, the lens clamp assembly 140 may also have the opposite orientation about the vertical axis of the main body 110 such that the actuating clip 150 engages the pocket 124a on the left side.

In the clamped position, as shown in FIG. 6C, the fixed protruding edge 146 and the movable protruding edge 156 are secured in the pockets 124a, 124b of the lens frame 120 such that ledges 149, 159 on tops of the protruding edges 146, 156 interfere with ledges inside the pockets 124a, 124b of the lens frame 120 (shown in the circled regions in FIG. 6C). FIG. 6C also shows the spring 154 biasing the actuating clip 150 into the pocket 124b in the clamped position and the lens stack 130 clamped and secured between the gasket 144 on the lens clamp 142 and the mounting surface 126. The resiliency of the gasket 144 causes a force to be applied by the ledges 149, 159 against the lens frame 120. When securely held in the clamping position, the lens clamp 142 prevents a gap from being formed between the lens 132 (e.g., the impact shield) and the mounting surface 126 of the lens frame 120, which could expose the user to possible debris or laser light.

As shown in FIGS. 6D and 6E, the releasable lens clamp 142 may be released from the clamped position by grasping the tab 158 and manually actuating the actuating clip 150 by rotating the actuating clip 150 counter-clockwise away from the lens frame 120 such that the movable protruding edge 156 moves out of the pocket 124b of the lens frame 120. The lens clamp 142 may then be angled upwardly away from the lens stack 130 and the lens frame 120 to remove the lead-in side of the lens clamp 142 from the pocket 124a. The user may then swap or replace lenses as needed.

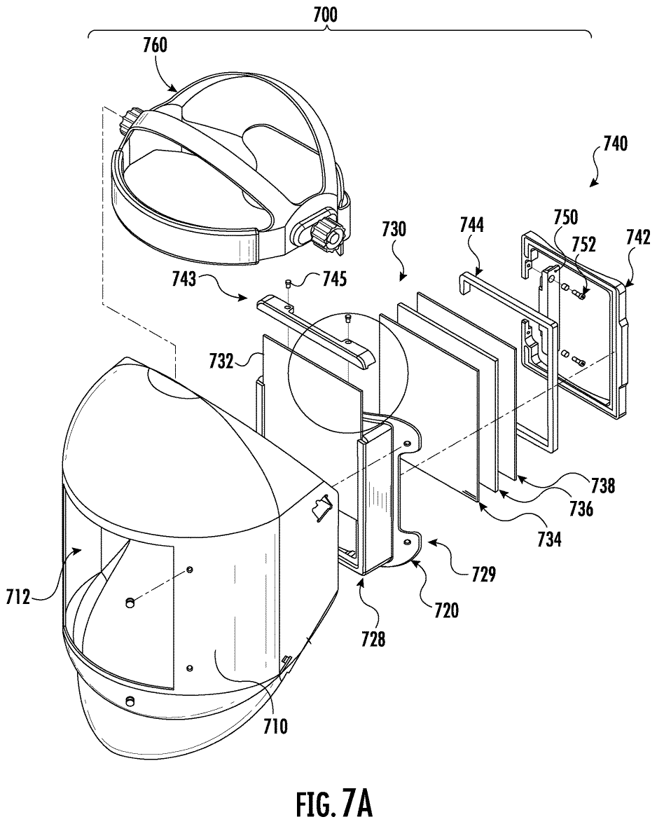

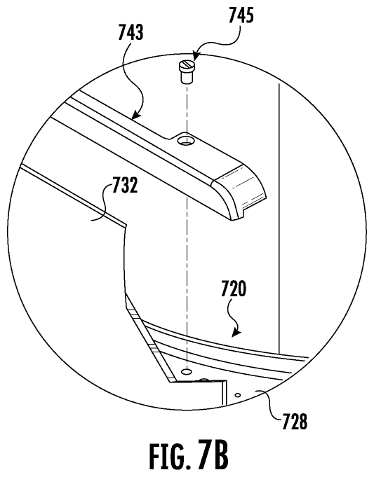

Referring to FIGS. 7A and 7B, another embodiment of a head-worn optical protection device 700 is described in greater detail. The head-worn optical protection device 700 includes a main body 710, a lens frame 720 positioned in a window 712 defined by the main body 710, and a lens stack 730 supported and framed by the lens frame 720. In this embodiment of the head-worn optical protection device 700, a lens clamp assembly 740 includes an inner lens clamp 742 that secures the lens stack 730 to the lens frame 720 inside the main body 710 and an outer lens clamp 743 that separately secures an impact shielding lens 132. The head-worn optical protection device 700 also includes headgear 760 secured to the main body 710 for mounting on the head of a user. The main body 710 and the headgear 760 may be similar to the embodiment described above.

The lens stack 730 may include one or more lenses and/or filters, for example, an IR protective lens 734, a filter 736, and a DIN-rated lens 738 (e.g., a DIN-rated weld shade), as described above. The impact shielding lens 732 may include, for example, a clear and completely transparent polycarbonate lens that provides impact resistance, as described above. A gasket 744, such as a foam gasket as described above, may be positioned between the lens stack 730 and the inner lens clamp 742.

In this embodiment of the head-worn optical protection device 700, the lens frame 720 includes an outer portion 728 in which the impact shielding lens 732 is secured by the outer lens clamp 743 and an inner portion 729 in which the lens stack 730 is secured by the inner lens clamp 742. As shown in greater detail in FIG. 7B, the outer lens clamp 743 may be in the form of a clamp bar secured to the outer portion 728 of the lens frame 720 by fasteners 745 such as screws. When the lens frame 720 is positioned in the window 712, the outer lens clamp 743 may be removed from the outside of the main body 710, for example, to replace the impact shielding lens 732. Thus, the impact shielding lens 732 may be removed and replaced, as needed, without removing the other lens in the lens stack 730.

The inner lens clamp 742 may be generally C-shaped and designed to engage and hold the lens stack 730, for example, similar to the lens clamp 142 described above. A clip 750 may be secured to the inner lens clamp 742, for example, using fasteners 752 such as screws. The inner lens clamp 742 and the clip 750 have protruding edges that engage pockets in the lens frame 720, similar to the lens clamp 142 and actuating clip 150 described above. In this embodiment, however, the clip 750 is secured to the inner lens clamp 742 without springs and without pivoting or rotating such that the user cannot release and easily remove the inner lens clamp 742. The inner lens clamp 742 thus allows the other lenses in the lens stack 730 to be removed but not as easily as using a releasable lens clamp with an actuating clip, as described in the embodiment above.

Accordingly, a lens clamp assembly used with the head-worn optical protection device, consistent with embodiments disclosed herein, advantageously allows one or more lenses and/or filters to be swapped out for different applications or replacement. In one embodiment, a lens clamp assembly facilitates actuation of the lens clamp using only one hand of the user by providing a actuating clip on one side of the clamp and securely holds the lens stack without adding much weight. In another embodiment, a lens clamp assembly includes separate lens clamps for an impact shielding lens and for other optical lenses to allow the impact shielding lens to be replaced without removing the other lenses.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

What is claimed is:

1. A head-worn optical protection device comprising:
a main body defining a window;
headgear for securing the main body to a head of a user;
a lens frame around the window in the main body, the lens frame defining first and second frame sides and first and second frame pockets located in the first and second frame sides, respectively;
a lens stack including at least one lens; and
a lens clamp assembly for releasably clamping the lens stack to the lens frame in the window of the main body, the lens clamp assembly comprising a lens clamp having first and second clamp sides engaging the first and second frame pockets for clamping the lens stack in the lens frame.

2. The head-worn optical protection device of claim 1, wherein the first clamp side of the lens clamp includes a fixed protruding edge extending from the first clamp side for insertion into one of the first frame pocket and the second frame pocket, and wherein the lens clamp assembly further comprises:
an actuating clip operably coupled to the second clamp side such that the actuating clip moves from an engaged position to an actuated position, wherein the actuating clip includes a movable protruding edge for releasably engaging another of the first frame pocket and the second frame pocket.

3. The head-worn optical protection device of claim 2 wherein the fixed protruding edge and the moving protruding edge have similar dimensions such that the clamp assembly is configured to engage the lens frame in different orientations about a vertical axis of the main body.

4. The head-worn optical protection device of claim 2 wherein the lens clamp assembly further comprises at least one fastening device securing the actuating clip to the lens clamp and at least one spring for biasing the actuating clip into the engaged position.

5. The head-worn optical protection device of claim 2 wherein the movable protruding edge of the actuating clip includes an angled surface.

6. The head-worn optical protection device of claim 2 wherein the actuating clip includes a tab extending above the movable protruding edge for gripping by the user.

7. The head-worn optical protection device of claim 2 wherein the actuating clip is configured to rotate relative to the lens clamp between the engaged position and the actuated position.

8. The head-worn optical protection device of claim 1 further comprising a resilient gasket positioned between the lens clamp and the lens stack.

9. The head-worn optical protection device of claim 3 wherein the resilient gasket is made of foam.

10. The head-worn optical protection device of claim 1 wherein the lens frame is mounted in the main body.

11. The head-worn optical protection device of claim 1 wherein the lens stack includes at least an impact shielding lens, an IR protective lens, and a DIN rated lens.

12. The head-worn optical protection device of claim 11 wherein the lens stack further includes a filter that filters off-angle light.

13. The head-worn optical protection device of claim 11 wherein the DIN rated lens includes a DIN rated lens having a DIN rating of 3-6.

14. The head-worn optical protection device of claim 11 wherein the impact shielding lens and the IR protective lens are bonded together.

15. The head-worn optical protection device of claim 1 wherein the lens frame includes an outer portion that protrudes from the main body through the window.

16. The head-worn optical protection device of claim 1 wherein the main body is made of carbon fiber.

17. The head-worn optical protection device of claim 1 wherein the main body and the lens frame are molded together from carbon fiber.

18. A head-worn optical protection device comprising:
a main body defining a window;
headgear for securing the main body to a head of a user;
a lens frame around the window in the main body, the lens frame having an outer portion extending through the window of the main body and an inner portion on an inside of the main body;
at least an impact shielding lens secured in the outer portion of the lens frame;
a lens stack secured in the inner portion of the lens frame; and
a lens clamp assembly configured to secure the impact shielding lens and the lens stack in the lens frame.

19. The head-worn optical protection device of claim 18, wherein the lens clamp assembly includes an outer lens clamp mounted to at least one side of the lens frame for securing the impact shielding lens in the outer portion of the lens frame separately from the lens stack.

20. The head-worn optical protection device of claim 19 wherein the outer lens clamp includes an outer clamp bar secured by threaded fasteners.

21. The head-worn optical protection device of claim 18 wherein the lens stack includes at least an IR protective lens and a DIN rated lens.

22. The head-worn optical protection device of claim 21 wherein the lens stack further includes a filter that filters off-angle light.

23. The head-worn optical protection device of claim 18, wherein the lens clamp assembly includes an inner lens clamp for securing the lens stack in the inner portion of the lens frame.

24. The head-worn optical protection device of claim 23 further comprising a resilient gasket positioned between the inner lens clamp and the lens stack.

25. The head-worn optical protection device of claim 23, wherein the inner lens clamp includes a releasable actuator configured to be actuated by a user for releasing the inner lens clamp.

26. The head-worn optical protection device of claim 23, wherein the inner lens clamp includes an inner clamp bar secured by threaded fasteners.

27. The head-worn optical protection device of claim 23, wherein the lens frame defines first and second frame sides and first and second pockets located in the first and second frame sides, respectively, wherein the inner lens clamp includes protruding edges that engage the first and second pockets to secure the plurality of lenses in the rear portion of the lens frame.
